(19) United States
(12) Patent Application Publication
(10) Pub. No.: US 2012/0157846 A1
(43) Pub. Date: Jun. 21, 2012

(54) ULTRASONIC DIAGNOSTIC SYSTEM AND PORTABLE ULTRASONIC DIAGNOSTIC APPARATUS THEREOF

(75) Inventors: Pai-Chi LI, Taipei (TW); Yen-Feng Li, Taipei (TW)

(73) Assignee: Pai-Chi Li, Taipei (TW)

(21) Appl. No.: 13/071,856

(22) Filed: Mar. 25, 2011

(30) Foreign Application Priority Data
Dec. 16, 2010 (TW) 099144239

(51) Int. Cl. A61B 8/14

(52) U.S. Cl. 600/443

(57) ABSTRACT

An exemplary ultrasonic diagnostic system includes a portable ultrasonic diagnostic apparatus and a cloud computing system. The portable ultrasonic diagnostic apparatus includes an ultrasonic sensing unit, a front-end circuit, a data compression unit and a communication unit. The ultrasonic sensing unit is configured to sense a signal reflected from a measured object. The front-end circuit is configured to perform a pre-processing on an output of the ultrasonic sensing unit. The data compression unit is configured to receive an output of the front-end circuit and determine whether to perform a data compression process on the output of the front-end circuit according to a selected result. The communication unit is configured to receive an output of the data compression unit and further transmit the output of the data compression unit to the cloud computing system.

### FIELD OF THE INVENTION

The present invention generally relates to ultrasonic technologies, and more particularly to an ultrasonic diagnostic system and a portable ultrasonic diagnostic apparatus thereof.

### BACKGROUND OF THE INVENTION

Since ultrasonic diagnosis is a type of imaging method without side effect to human body and an ultrasonic diagnostic system has several advantages of cost effectiveness, high convenience, widespread applications and real-time capabilities, the ultrasonic diagnostic system is commonly equipped in most hospitals and also is an efficient tool for doctors to inspect patients.

However, the conventional ultrasonic diagnostic system usually is a stand-alone system having complex design and relatively large size, so that the conventional ultrasonic diagnostic system has to be arranged at a fixed position for use. Today, even though portable systems are available, its size still limits its clinical applications. This disadvantage brings inconvenience to both hospitals and patients.

### SUMMARY OF THE INVENTION

Therefore, an objective of the present invention is to provide a portable ultrasonic diagnostic apparatus adapted for use together with a cloud computing system (i.e., a remote server).

Another objective of the present invention is to provide an ultrasonic diagnostic system comprising the portable ultrasonic diagnostic apparatus.

More specifically, a portable ultrasonic diagnostic apparatus in accordance with an embodiment of the present invention comprises an ultrasonic sensing unit, a front-end circuit, a data compression unit and a communication unit. The ultrasonic sensing unit is configured (i.e., structured and arranged) to sense a signal reflected from a measured object. The front-end circuit is configured to perform a preprocessing process on an output of the ultrasonic sensing unit. The data compression unit is configured to receive an output of the front-end circuit and determine whether to perform a data compression processing on the output of the front-end circuit according to a selected result. The communication unit is configured to receive an output of the data compression unit and further transmit the output of the data compression unit to a cloud computing system.

An ultrasonic diagnostic system in accordance with another embodiment of the present invention comprises the above mentioned portable ultrasonic diagnostic apparatus and a cloud computing system. The cloud computing system is configured to receive a signal corresponding to the output of the data compression unit transmitted from the communication unit of the portable ultrasonic diagnostic apparatus, perform a data extraction/decompression processing on the received signal and thereby rebuild an ultrasonic image.

In one embodiment, the above mentioned cloud computing system is further for performing at least one of image forming operation, image analysis operation, image archiving operation and computer aided diagnosis operation.

In one embodiment, the above mentioned data compression processing and the data decompression processing both are performed based on a lossy compression method, or a lossless compression method instead.

In one embodiment, the above mentioned cloud computing system comprises a computer apparatus installed with a back-end processing software. The back-end processing software is configured to perform the data decompression processing on the signal received by the cloud computing system and thereby rebuild the ultrasonic image.

In one embodiment, the above mentioned back-end processing software is executed by a central processing unit (CPU) or a graphic processing unit (GPU) of the computer apparatus.

In one embodiment, the above mentioned communication unit is linked to the cloud computing system in a wired manner or in a wireless manner.

In one embodiment, the above mentioned ultrasonic sensing unit comprises at least one ultrasonic sensing probe.

In one embodiment, the above mentioned portable ultrasonic diagnostic apparatus further comprises a user input interface and a control unit. The control unit is configured to receive a command entered by a user through the user input interface, and thereby generate a control signal according to the command, so that the data compression unit obtains the selected result according to the control signal.

In one embodiment, the above mentioned control unit further is configured to control the data compression unit to select a required data compression format according to a command entered by the user.

In summary, the ultrasonic diagnostic system of the present invention primarily is constructed by the portable ultrasonic diagnostic apparatus and the cloud computing system, and the portable ultrasonic diagnostic apparatus primarily is comprised of the ultrasonic sensing unit, the front-end circuit, the data compression unit and the communication unit. Because the inspection result generated by the portable ultrasonic diagnostic apparatus associated with a measured object would be transmitted by the communication unit to the cloud computing system for further processing, the ultrasonic diagnostic system of the present invention is not necessary to be set at a fixed location. Moreover, because the data compression unit of the portable ultrasonic diagnostic apparatus determines whether to perform a data compression processing on the output of the front-end circuit according to the selected result, a user can change the selected result according to actual requirement and thereby change the amount of data transmission between the portable ultrasonic diagnostic apparatus and the cloud computing system. As a result, an image reconstruction rate of the cloud computing system rebuilding the ultrasonic image can be improved.

### BRIEF DESCRIPTION OF THE DRAWING

The above objects and advantages of the present invention will become more readily apparent to those ordinarily skilled in the art after reviewing the following detailed description and accompanying drawing, in which:

FIG. 1 is a schematic block diagram of an ultrasonic diagnostic system in accordance with an embodiment of the present invention.

### DETAILED DESCRIPTION OF PREFERRED EMBODIMENTS

The present invention will now be described more specifically with reference to the following embodiments. It is to be noted that the following descriptions of preferred embodiments of this invention are presented herein for purpose of illustration and description only. It is not intended to be exhaustive or to be limited to the precise form disclosed.

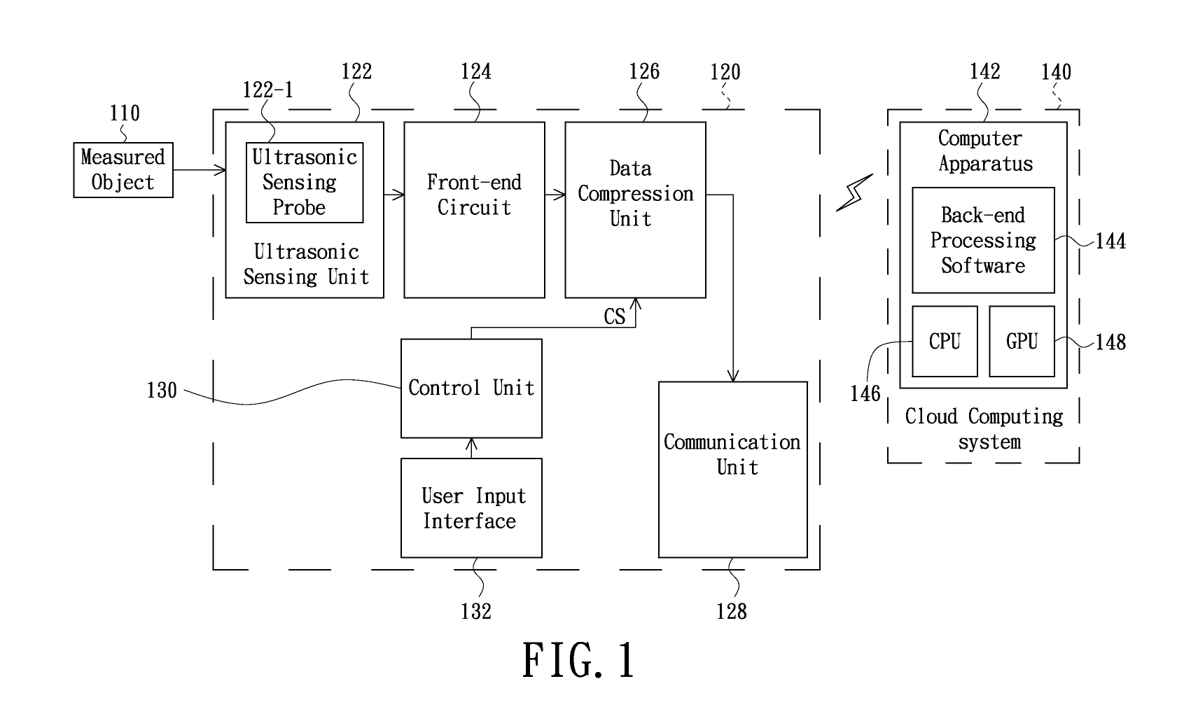

FIG. 1 is a schematic block diagram of an ultrasonic diagnostic system in accordance with an embodiment of the present invention. As depicted in FIG. 1, the ultrasonic diagnostic system comprises a portable ultrasonic diagnostic apparatus 120 and a cloud computing system 140. The portable ultrasonic diagnostic apparatus 120 primarily comprises an ultrasonic sensing unit 122, a front-end circuit 124, a data compression unit 126 and a communication unit 128. The ultrasonic sensing unit 122 is configured (i.e., structured and arranged) to sense signals reflected from a measured object 110. The front-end circuit 124 is configured to perform a pre-processing on an output of the ultrasonic sensing unit 122. The data compression unit 126 is configured to receive an output of the front-end circuit 124 and determine whether to perform a data compression processing on the output of the front-end circuit 124 according to a selected result. Herein, the selected result generally is a result of determining the data compression unit 126 whether to perform the data compression processing. The communication unit 128 is configured to receive an output of the data compression unit 126 and further transmit the output of the data compression unit 126 to the cloud computing system 140.

Since the data compression unit 126 can determine whether to perform the data compression processing on the output of the front-end circuit 124 based on the selected result, the user can vary the selected result according to actual requirement, which facilitates to vary the amount of data transmission between the portable ultrasonic diagnostic apparatus 120 and the cloud computing system 140, so that an image reconstruction rate of the cloud computing system 140 rebuilding the ultrasonic image can be improved consequently. For example, during a general scanning/inspecting, the data compression unit 126 can compress obtained data to effectively reduce the amount of data transmission between the portable ultrasonic diagnostic apparatus 120 and the cloud computing system 140, so that the image reconstruction rate of the cloud computing system 140 is increased. In another aspect, after carrying out preliminary inspection, if there is a requirement of detailed inspection, the data compression unit 126 might not perform the data compression processing and the uncompressed data is directly transmitted to the cloud computing system 140 by the communication unit 128, so that the distortion caused by data compression would be avoided.

In a preferred embodiment, the portable ultrasonic diagnostic apparatus 120 may further comprise a control unit 130 and a user input interface 132. The user input interface 132 is configured to allow a user to input a command. The control unit 130 is configured to generate a control signal CS based on the command entered to the user input interface 132 by the user. As a result, the data compression unit 126 can obtain the selected result according to the control signal CS. Moreover, in an advanced design, the control unit 130 further can be configured to control the data compression unit 126 to select a required data compression format, such as JPEG (joint photographic experts group), JPEG2000 or other data compression format, facilitating the ultrasonic image rebuilt by the cloud computing system 140 has an acceptable image quality.

Moreover, the ultrasonic sensing unit 122 comprises at least one ultrasonic sensing probe 122-1. If the ultrasonic sensing unit 122 comprises more than one (i.e., multiple) ultrasonic sensing probes, these ultrasonic sensing probes can be arranged in a form of one-dimensional array or in a form of two-dimensional array instead. In addition, the connection between the communication unit 128 and the cloud computing system 140 can be achieved in a wired manner (such as USB3.0), or in a wireless manner (such as Wi-Fi or 3G) instead. For example, the communication unit 128 can be firstly linked to Internet via a wireless network technology, and the internet then is linked to the cloud computing system 140 to establish the connection between the communication unit 128 and the cloud computing system 140 consequently. Owing to the progress of transmission technology, a data transmission rate of either the wired technology or the wireless technology nowadays has reached the level of Gbps (giga bit per second), it is absolutely feasible to transmit image data between the communication unit 128 and the cloud computing system 140 in wired manner or in wireless manner.

The cloud computing system 140 is configured to receive the signal outputted from the communication unit 128 of the portable ultrasonic diagnostic apparatus 120, perform a data extraction/decompression processing on the received signal and thereby rebuild/reconstruct an ultrasonic image based on the decompressed signal. In a preferred embodiment, the cloud computing system 140 further is configured to perform at least one of image forming operation, image analysis operation, image archiving operation and computer aided diagnosis operation.

In particular, the cloud computing system 140 comprises a computer apparatus 142 which is installed with a back-end processing software 144. The back-end processing software 144 is configured to perform the data decompression processing on the signal received by the cloud computing system 140 and thereby rebuild the ultrasonic image. In a preferred embodiment, the back-end processing software 144 further is configured to perform at least one of image forming operation, image analysis operation, image archiving operation and computer aided diagnosis operation. Moreover, the back-end processing software 144 can be executed by a CPU (central processing unit) 146 or a GPU (graphic processing unit) 148 of the computer system 142.

It is indicated that, the aforementioned data compression processing and the data decompression processing both can be performed based on either a lossy compression method or a lossless compression method.

To sum up, the ultrasonic diagnostic system in accordance with each of the various embodiments of the present invention primarily is constructed by a portable ultrasonic diagnostic apparatus and a cloud computing system, and the portable ultrasonic diagnostic apparatus primarily is comprised of an ultrasonic sensing unit, a front-end circuit, a data compression unit and a communication unit. Because the inspection signals generated by the portable ultrasonic diagnostic apparatus associated with a measured object would be by the communication unit transmitted to the cloud computing system for further processing, the ultrasonic diagnostic system of the present invention no longer is necessary to be set at a fixed location like the prior art. Moreover, because the data compression unit of the portable ultrasonic diagnostic apparatus determines whether to perform a data compression processing on the output of the front-end circuit according to the selected result, a user can vary the selected result according to actual requirement and thereby vary the amount of data transmission between the portable ultrasonic diagnostic apparatus and the cloud computing system, an image reconstruction rate of the cloud computing system rebuilding the ultrasonic image is improved consequently.

While the invention has been described in terms of what is presently considered to be the most practical and preferred embodiments, it is to be understood that the invention needs not be limited to the disclosed embodiment. On the contrary, it is intended to cover various modifications and similar arrangements included within the spirit and scope of the appended claims which are to be accorded with the broadest interpretation so as to encompass all such modifications and similar structures.

What is claimed is:

1. A portable ultrasonic diagnostic apparatus comprising:
an ultrasonic sensing unit, for sensing a signal reflected from a measured object;
a front-end circuit, for performing a pre-processing on an output of the ultrasonic sensing unit;
a data compression unit, for receiving an output of the front-end circuit and determining whether to perform a data compression processing on the output of the front-end circuit according to a selected result; and
a communication unit, for receiving an output of the data compression unit and transmitting the output of the data compression unit to a cloud computing system.

2. The portable ultrasonic diagnostic apparatus according to claim 1, wherein a signal corresponding to the output of the data compression unit and transmitted from the communication unit is received the cloud computing system, performed with a data decompression processing by the clouding computing system and thereby rebuilt to be an ultrasonic image by the clouding computing system.

3. The portable ultrasonic diagnostic apparatus according to claim 2, wherein at least one of image forming operation, image analysis operation, image archiving operation and computer aided diagnosis operation is further performed by the clouding computing system based on the ultrasonic image.

4. The portable ultrasonic diagnostic apparatus according to claim 2, wherein the data compression processing and the data decompression processing both are performed based on one of a lossy compression method and a lossless compression method.

5. The portable ultrasonic diagnostic apparatus according to claim 1, wherein the data compression processing is performed based on one of a lossy compression method and a lossless compression method.

6. The portable ultrasonic diagnostic apparatus according to claim 1, wherein the communication unit is linked to the cloud computing system in one of a wired manner and a wireless manner.

7. The portable ultrasonic diagnostic apparatus according to claim 1, wherein the ultrasonic sensing unit comprises at least an ultrasonic sensing probe.

8. The portable ultrasonic diagnostic apparatus according to claim 1, further comprising:
a user input interface; and
a control unit, for receiving a command entered by a user through the user input interface and thereby generating a control signal to allow the data compression unit to obtain the selected result according to the control signal.

9. The portable ultrasonic diagnostic apparatus according to claim 8, wherein the control unit further is for controlling the data compression unit to select a data compression format according to a command entered by the user.

10. An ultrasonic diagnostic system comprising:
a portable ultrasonic diagnostic apparatus, comprising:
an ultrasonic sensing unit, for sensing a signal reflected from a measured object;
a front-end circuit, for performing a pre-processing on an output of the ultrasonic sensing unit;
a data compression unit, for receiving an output of the front-end circuit and determining whether to perform a data compression processing on the output of the front-end circuit according to a selected result; and
a communication unit, for receiving and transmitting an output of the data compression unit; and
a cloud computing system, for receiving a signal corresponding to the output of the data compression unit transmitted from the communication unit, performing a data decompression processing on the received signal and thereby rebuilding an ultrasonic image.

11. The ultrasonic diagnostic system according to claim 10, wherein the cloud computing system is further for performing at least one of image forming operation, image analysis operation, image archiving operation and computer aided diagnosis operation.

12. The ultrasonic diagnostic system according to claim 10, wherein the data compression process and the data decompression processing both are performed based on one of a lossy compression method and a lossless compression method.

13. The ultrasonic diagnostic system according to claim 10, wherein the cloud computing system comprises a computer apparatus installed with a back-end processing software, the back-end processing software performs the data decompression processing on the received signal of the cloud computing system and thereby rebuilds the ultrasonic image.

14. The ultrasonic diagnostic system according to claim 13, wherein the back-end processing software is executed by a central processing unit or a graphic processing unit of the computer apparatus.

15. The ultrasonic diagnostic system according to claim 10, wherein the communication unit is linked to the cloud computing system in one of a wired manner or a wireless manner.

16. The ultrasonic diagnostic system according to claim 10, wherein the ultrasonic sensing unit comprises at least one ultrasonic sensing probe.

17. The ultrasonic diagnostic system according to claim 10, wherein the portable ultrasonic diagnostic apparatus further comprises:
a user input interface; and
a control unit, for receiving a command entered by a user through the user input interface and thereby generating a control signal to allow the data compression unit to obtain the selected result according to the control signal.

18. The ultrasonic diagnostic system according to claim 17, wherein the control unit further is for controlling the data compression unit to select a data compression format according to a command entered by the user.
